(19) United States
(12) Patent Application Publication
(10) Pub. No.: US 2015/0342673 A1
(43) Pub. Date: Dec. 3, 2015

(54) NERVE MODULATION DEVICES WITH COOLING CAPABILITIES

(71) Applicant: BOSTON SCIENTIFIC SCIMED, INC., MAPLE GROVE, MN (US)

(72) Inventors: ROBERT N. SQUIRE, MAPLE GROVE, MN (US); ADAM J. ROYER, BROOKLYN PARK, MN (US); DEREK C. SUTERMEISTER, HAM LAKE, MN (US); CASS A. HANSON, ST. PAUL, MN (US); JEFFREY S. LINDQUIST, MAPLE GROVE, MN (US)

(73) Assignee: BOSTON SCIENTIFIC SCIMED, INC., MAPLE GROVE, MN (US)

(21) Appl. No.: 14/723,000

(22) Filed: May 27, 2015

Related U.S. Application Data
(60) Provisional application No. 62/003,991, filed on May 28, 2014.

(51) Int. Cl. A61B 18/14

(52) U.S. Cl. CPC A61B 18/1492; A61B 2018/1465; A61B 2018/00791

(57) ABSTRACT

Medical devices and methods and making and using medical devices are disclosed. An example medical device may include an elongate shaft having a distal region. An expandable member may be coupled to the distal region. The expandable member may be capable of shifting between a first configuration and an expanded configuration. The expandable member may have a distal portion, a proximal portion, and a body portion disposed between the distal portion and the proximal portion. The body portion may have a projection formed therein that projects radially outward from the body portion. A flexible electrode assembly may be coupled to the projection.

### CROSS-REFERENCE TO RELATED APPLICATIONS

This application claims priority under 35 U.S.C. §119 to U.S. Provisional Application Ser. No. 62/003,991, filed May 28, 2014, the entirety of which is incorporated herein by reference.

### TECHNICAL FIELD

The present disclosure pertains to medical devices, and methods for manufacturing medical devices. More particularly, the present disclosure pertains to elongated intracorporeal medical devices including a tubular member connected with other structures, and methods for manufacturing and using such devices.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. An example medical device may include an elongate shaft having a distal region. An expandable member may be coupled to the distal region. The expandable member may be capable of shifting between a first configuration and an expanded configuration. The expandable member may have a distal portion, a proximal portion, and a body portion disposed between the distal portion and the proximal portion. The body portion may have a projection formed therein that projects radially outward from the body portion. A flexible electrode assembly may be coupled to the projection.

In a first example, a medical device is disclosed. The medical device includes an elongate shaft having a distal region. An expandable member is coupled to the distal region. The expandable member is capable of shifting between a first configuration and an expanded configuration. The expandable member has a distal portion, a proximal portion, and a body portion disposed between the distal portion and the proximal portion. The body portion has a projection formed therein that projects radially outward from the body portion. A flexible electrode assembly is coupled to the projection.

Alternatively or additionally to any of the embodiments above, the expandable member includes an expandable balloon.

Alternatively or additionally to any of the embodiments above, the projection includes a circumferential ring disposed along the expandable member.

Alternatively or additionally to any of the embodiments above, the projection includes a helical ridge disposed along the expandable member.

Alternatively or additionally to any of the embodiments above, the medical device further comprises one or more additional projections.

Alternatively or additionally to any of the embodiments above, at least some of the one or more additional projections include an additional flexible electrode assembly.

Alternatively or additionally to any of the embodiments above, the flexible electrode assembly includes a monopolar electrode.

Alternatively or additionally to any of the embodiments above, the flexible electrode assembly includes a pair of bipolar electrodes.

Alternatively or additionally to any of the embodiments above, the flexible electrode assembly includes a temperature sensor.

Alternatively or additionally to any of the embodiments above, a section of the expandable member is removed to define the projection.

Alternatively or additionally to any of the embodiments above, the projection is defined by a layer of material disposed on the expandable member.

Another example medical device may include an elongate shaft having a distal region. An expandable balloon may be coupled to the distal region. The balloon may be capable of shifting between a first configuration and an expanded configuration. The balloon may have a distal cone portion, a proximal cone portion, and a body portion disposed between the distal cone portion and the proximal cone portion. The body portion may have a projection region therein that projects radially outward from the body portion. A flexible electrode assembly may be coupled to the projection region.

Alternatively or additionally to any of the embodiments above, the projection region includes one or more circumferential rings disposed along the balloon.

Alternatively or additionally to any of the embodiments above, the projection region includes a helical ridge disposed along the balloon.

Alternatively or additionally to any of the embodiments above, the medical device further comprises one or more additional projection regions disposed along the body portion of the balloon.

Alternatively or additionally to any of the embodiments above, at least some of the one or more additional projection regions include an additional flexible electrode assembly.

Alternatively or additionally to any of the embodiments above, the flexible electrode assembly includes one or more monopolar electrodes.

Alternatively or additionally to any of the embodiments above, the flexible electrode assembly includes one or more pairs of bipolar electrodes.

Alternatively or additionally to any of the embodiments above, the flexible electrode assembly includes a temperature sensor.

An example method for ablating tissue adjacent to a renal artery may include advancing an ablation catheter through a blood vessel to a position adjacent to a renal artery. The ablation catheter may include an elongate shaft having a distal region. An expandable balloon may be coupled to the distal region. The balloon may be capable of shifting between a first configuration and an expanded configuration. The balloon may have a distal cone portion, a proximal cone portion, and a body portion disposed between the distal cone portion and the proximal cone portion. The body portion may have a projection region therein that projects radially outward from the body portion. A flexible electrode assembly may be coupled to the projection region. The method may also include shifting the balloon to the expanded configuration and activating the electrode assembly.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a schematic view of an example sympathetic nerve ablation device;

FIG. 2 is a perspective view of an example expandable member of a sympathetic nerve ablation device;

FIG. 3 is a partial top view of the expandable member of FIG. 2 in an unrolled or flat configuration;

FIG. 4 is a perspective view of a portion of an example sympathetic nerve ablation device;

FIG. 5 is a perspective view of a portion of an example sympathetic nerve ablation device;

FIG. 6 is a perspective view of a portion of an example sympathetic nerve ablation device; and

FIG. 7 is a perspective view of a portion of an example sympathetic nerve ablation device.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

Certain treatments are aimed at the temporary or permanent interruption or modification of select nerve function. In some embodiments, the nerves may be sympathetic nerves. One example treatment is renal nerve ablation, which is sometimes used to treat conditions such as or related to hypertension, congestive heart failure, diabetes, or other conditions impacted by high blood pressure or salt retention. The kidneys produce a sympathetic response, which may increase the undesired retention of water and/or sodium. The result of the sympathetic response, for example, may be an increase in blood pressure. Ablating some of the nerves running to the kidneys (e.g., disposed adjacent to or otherwise along the renal arteries) may reduce or eliminate this sympathetic response, which may provide a corresponding reduction in the associated undesired symptoms (e.g., a reduction in blood pressure).

Some embodiments of the present disclosure relate to a power generating and control apparatus, often for the treatment of targeted tissue in order to achieve a therapeutic effect. In some embodiments, the target tissue is tissue containing or proximate to nerves. In other embodiments, the target tissue is sympathetic nerves, including, for example, sympathetic nerves disposed adjacent to blood vessels. In still other embodiments the target tissue is luminal tissue, which may further comprise diseased tissue such as that found in arterial disease.

In some embodiments of the present disclosure, the ability to deliver energy in a targeted dosage may be used for nerve tissue in order to achieve beneficial biologic responses. For example, chronic pain, urologic dysfunction, hypertension, and a wide variety of other persistent conditions are known to be affected through the operation of nervous tissue. For example, it is known that chronic hypertension that may not be responsive to medication may be improved or eliminated by disabling excessive nerve activity proximate to the renal arteries. It is also known that nervous tissue does not naturally possess regenerative characteristics. Therefore it may be possible to beneficially affect excessive nerve activity by disrupting the conductive pathway of the nervous tissue. When disrupting nerve conductive pathways, it is particularly advantageous to avoid damage to neighboring nerves or organ tissue. The ability to direct and control energy dosage is well-suited to the treatment of nerve tissue. Whether in a heating or ablating energy dosage, the precise control of energy delivery as described and disclosed herein may be directed to the nerve tissue. Moreover, directed application of energy may suffice to target a nerve without the need to be in exact contact, as would be required when using a typical ablation probe. For example, eccentric heating may be applied at a temperature high enough to denature nerve tissue without causing ablation and without requiring the piercing of luminal tissue. However, it may also be desirable to configure the energy delivery surface of the present disclosure to pierce tissue and deliver ablating energy similar to an ablation probe with the exact energy dosage being controlled by a power control and generation apparatus.

In some embodiments, efficacy of the denervation treatment can be assessed by measurement before, during, and/or after the treatment to tailor one or more parameters of the treatment to the particular patient or to identify the need for additional treatments. For instance, a denervation system may include functionality for assessing whether a treatment has caused or is causing a reduction in neural activity in a target or proximate tissue, which may provide feedback for adjusting parameters of the treatment or indicate the necessity for additional treatments.

Many of the devices and methods described herein are discussed relative to renal nerve ablation and/or modulation. However, it is contemplated that the devices and methods may be used in other treatment locations and/or applications where sympathetic nerve modulation and/or other tissue modulation including heating, activation, blocking, disrupting, or ablation are desired, such as, but not limited to: blood vessels, urinary vessels, or in other tissues via trocar and cannula access. For example, the devices and methods described herein can be applied to hyperplastic tissue ablation, cardiac ablation, pain management, pulmonary vein isolation, pulmonary vein ablation, tumor ablation, benign prostatic hyperplasia therapy, nerve excitation or blocking or ablation, modulation of muscle activity, hyperthermia or other warming of tissues, etc. The disclosed methods and apparatus can be applied to any relevant medical procedure, involving both human and non-human subjects. The term modulation refers to ablation and other techniques that may alter the function of affected nerves and other tissue.

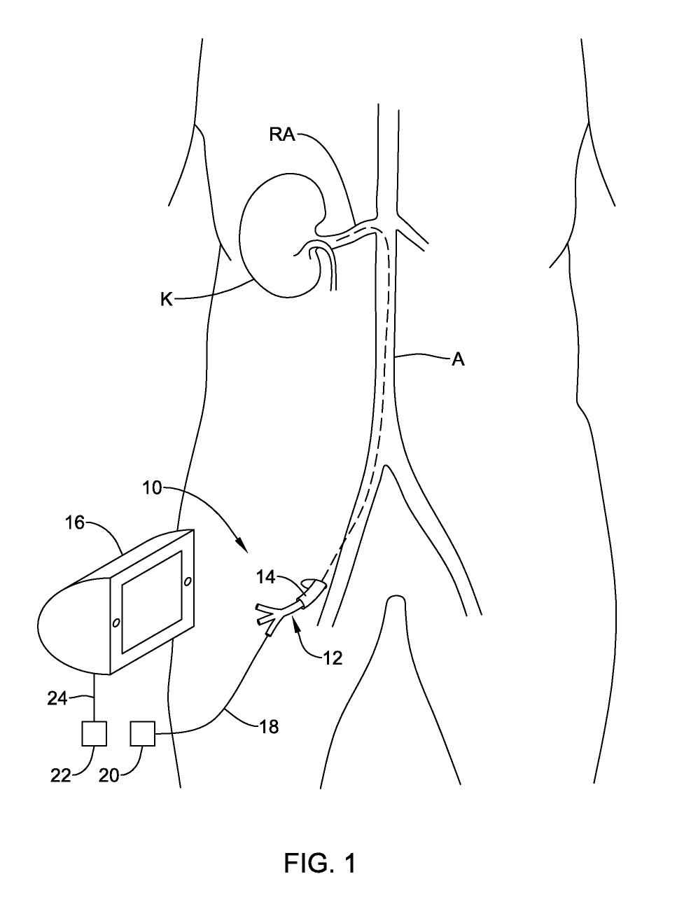

FIG. 1 is a schematic view of an example sympathetic nerve ablation system 10. System 10 may include a sympathetic nerve ablation device 12. Sympathetic nerve ablation device 12 may be used to ablate nerves (e.g., renal nerves) disposed adjacent to the kidney K (e.g., renal nerves disposed about a renal artery RA). In use, sympathetic nerve ablation device 12 may be advanced through a blood vessel such as the aorta A to a position within the renal artery RA. This may include advancing sympathetic nerve ablation device 12 through a guide sheath or catheter 14. When positioned as desired, sympathetic nerve ablation device 12 may be activated to activate one or more electrodes (not shown). This may include operatively coupling sympathetic nerve ablation device 12 to a control unit 16, which may include an RF generator, so as to supply the desired activation energy to the electrodes. For example, sympathetic nerve ablation device 12 may include a wire or conductive member 18 with a first connector 20 that can be connected to a second connector 22 on the control unit 16 and/or a wire 24 coupled to the control unit 16. In at least some embodiments, the control unit 16 may also be utilized to supply/receive the appropriate electrical energy and/or signal to activate one or more sensors disposed at or near a distal end of sympathetic nerve ablation device 12. When suitably activated, the one or more electrodes may be capable of ablating tissue (e.g., sympathetic nerves) as described below and the one or more sensors may be used to detect desired physical and/or biological parameters.

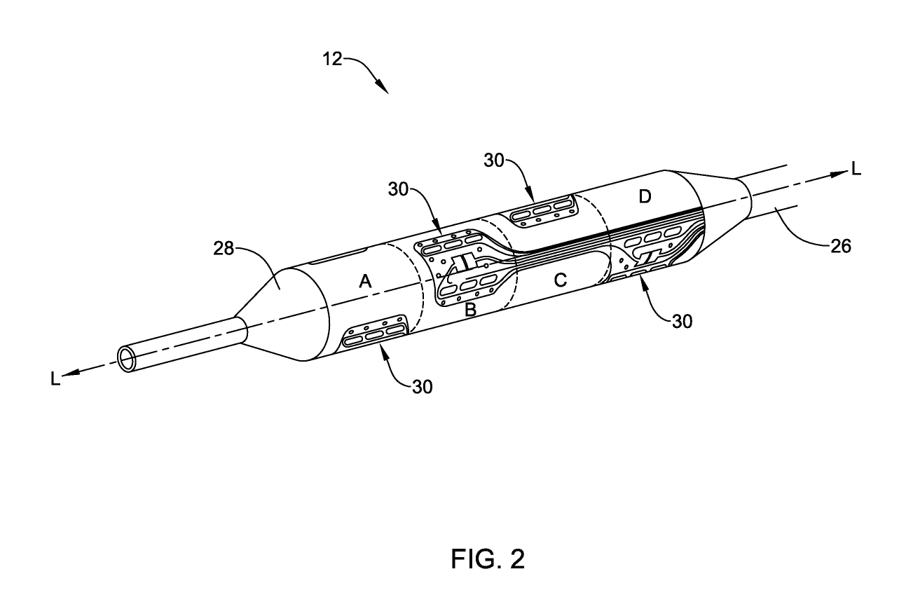

In some embodiments, sympathetic nerve ablation device 12 may include an elongate tubular member or catheter shaft 26, as shown in FIG. 2. In some embodiments, shaft 26 may be configured to be advanced to a target site over a guidewire, within guide sheath or catheter 14, or a combination thereof. An expandable member 28 may be disposed at, on, about, or near a distal region of shaft 26. Expandable member 28 may be a compliant or a non-compliant balloon, cage, mesh, plurality of struts, or the like. In some embodiments, expandable member 28 may be capable of shifting between an unexpanded configuration and an expanded configuration.

For example, as shown in FIG. 2, in some embodiments, one or more flexible electrode assemblies 30 may be arranged on expandable member 28, shown in an expanded state, according to a plurality of generally cylindrical treatment zones A-D. In other embodiments, expandable member 28 or other components of the treatment system may include additional electrode assemblies that are not in a treatment zone or are otherwise not used or configured to deliver a treatment energy.

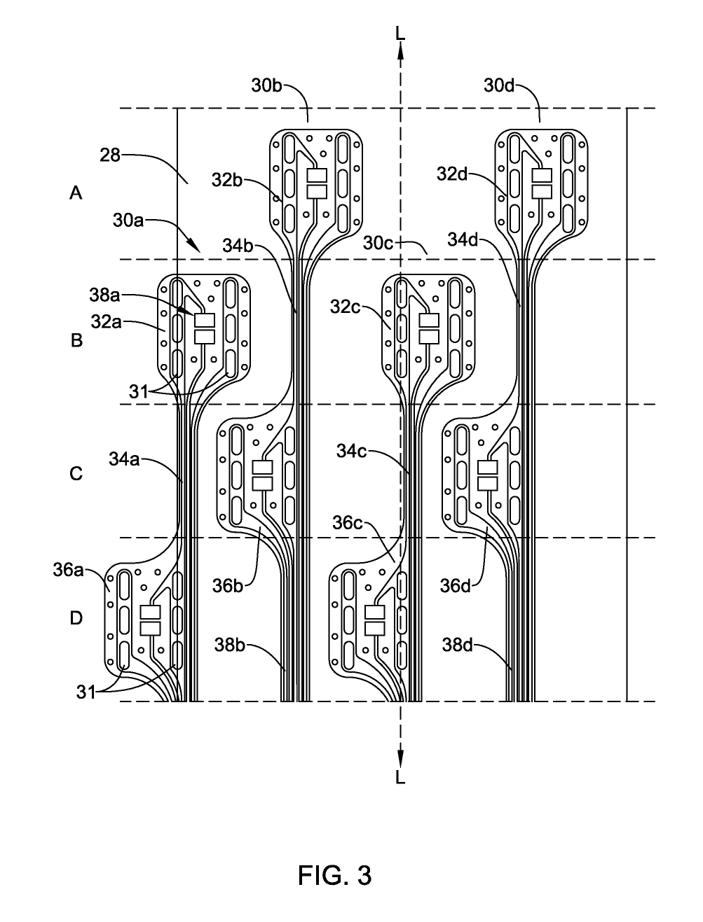

The treatment zones A-D and associated flexible electrode assemblies 30 are further illustrated in FIG. 3 (labeled as assemblies 30a-d), which is an “unrolled” depiction of a portion of expandable member 28. The treatment zones A-D may be longitudinally adjacent to one another along longitudinal axis L-L, and may be configured such that energy applied by the electrode assemblies create treatments that may or may not overlap. Each flexible electrode assembly 30a-d may include a number of structural features including a distal electrode pad 32a-d, intermediate tail 34a-d, proximal electrode pad 36a-d, proximal tail 38b/d (not shown for electrode pad assemblies 30a and 30c), and a temperature sensor like the one identified with number 38a (e.g., a thermistor, a thermocouple, or the like, which may be coupled to one or more distal electrode pads 32a-d and/or proximal electrode pads 36a-d). Some additional details regarding electrode assemblies 30a-30d may be found, for example, in U.S. Patent Application Pub. No. US 2013/0165926, the entire disclosure of which is herein incorporated by reference.

Distal electrode pads 32a-d and/or proximal electrode pads 36a-d may include one or more electrodes 31. In some embodiments, electrodes 31 may include one or more monopolar electrodes. In these embodiments, a return electrode pad (not shown) may be used and may be disposed along the skin of the patient. In some of these and in other embodiments, electrodes 31 may include one or more pairs of bipolar electrodes.

In some embodiments, the energy applied by electrode assemblies, such as flexible electrode assemblies 30a-d shown in FIG. 3, may create lesions that may overlap longitudinally, circumferentially, and/or in other ways. Longitudinally and/or circumferentially overlapping lesions may be effective to modulate nerves. However, in some instances it may be desirable for the lesions to have less circumferential and/or longitudinal overlap. This may help to reduce damage to the blood vessels, reduce long term stenotic effects adjacent the treatment site, or the like.

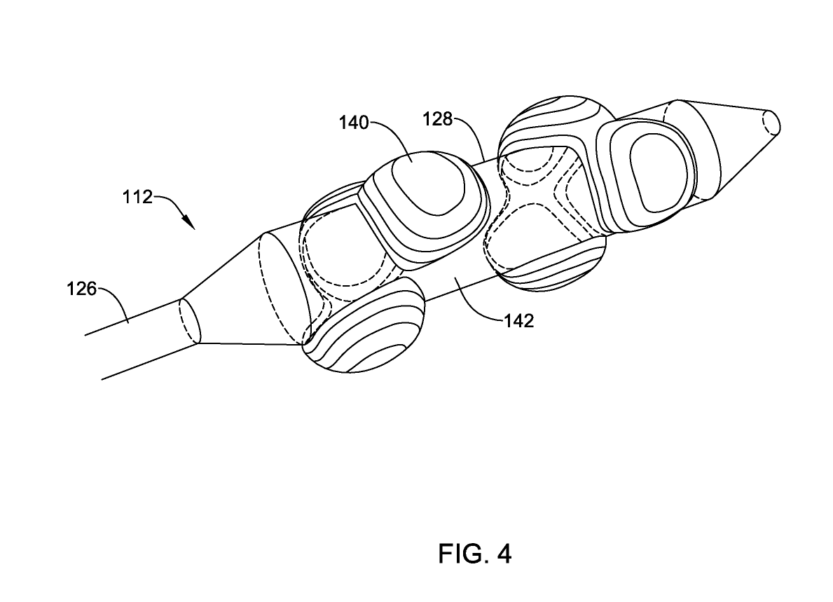

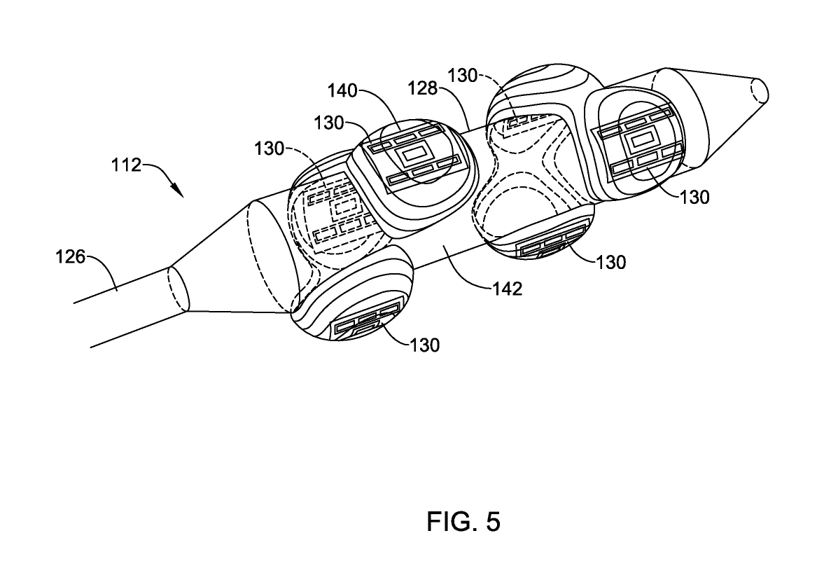

FIGS. 4-5 illustrate an example device 112, similar in form and function to other devices disclosed herein, that is designed to reduce the amount of longitudinal and/or circumferential overlap of lesions. Device 112 may include shaft 126 and balloon 128. Balloon 128 may include one or more radial projections 140. Projections 140 may be designed to project outward from the outer surface of balloon 128 so that, for example, projections 140 may contact the wall of a blood vessel and portions of balloon 128 adjacent to projections 140 may be spaced from wall of the blood vessel. The “spaced away”, or radially extended portions of balloon may define one or more cooling pathways 142. Cooling pathways 142 may be understood to be regions along the outer surface of balloon 128 (adjacent to projections 140) that can be spaced from the wall of a blood vessel so that fluid (e.g., blood) may flow along balloon 128. These regions are designed to have little or no apposition to the vessel wall and thus little or no heat transfer to the vessel wall in those regions. The flowing of blood/fluid may allow for higher temperature regions that may begin to form adjacent to electrode assemblies 130 to be “cooled”. In at least some embodiments, flowing blood may provide the cooling effect along cooling pathways 142. In some of these and in other embodiments, a cooling fluid may be infused by device 112 (or another device) that may flow along cooling pathways 142 so as to provide additional cooling.

Some balloons suitable for use with medical devices may have a central body portion, proximal and/or distal cone sections (which may have a generally conical shape), and proximal and/or distal waist sections where the balloon bonds to the catheter shaft(s). Projections 140 may be disposed along a suitable region of balloon 128. This may include along the body portion of balloon 128. However, embodiments are contemplated where projections 140 may be disposed along other portions of balloon 128 such as the proximal/distal cone sections.

A suitable number of projections 140 may be used with balloon 128. For example, balloon 128 may include one, two, three, four, five, six, seven, eight, nine, ten, or more projections 140. Projections 140 may be disposed along balloon 128 in a suitable pattern/arrangement. This may include patterns where differing projections 140 are longitudinally and/or circumferentially offset from one another.

Projections 140 may be formed by adding one or more layers of material onto the outer surface of balloon 128. Alternatively, projections 140 may be formed by removing portions of balloon 128 so that areas where material is not removed define projections 140. In still other embodiments, projections 140 may be defined through the process of forming balloon (e.g., molding). For example, the shape and form of projections 140 may be defined through a modified balloon molding process that results in balloon 128 having the desired shape/projections 140.

In at least some embodiments, flexible electrode assemblies 130 (shown schematically in FIG. 5, but may be similar to other electrode assemblies disclosed herein) may be coupled to balloon 128 as shown in FIG. 5. This may include disposing flexible electrode assemblies 130 at or otherwise along projections 140. This allows electrode assemblies 130 to come into contact with target tissue while allowing cooling pathways 142 to be positioned adjacent to projections 140/assemblies 130 to reduce the spread of heat adjacent to assemblies 130. This may allow for more discrete lesions to be formed by activating assemblies 130 and/or otherwise reduce longitudinal/circumferential residual heat overlap. In some instances, flexible electrode assemblies 130 may be disposed in their entirety along projections 140 (e.g., the main body of an entire flexible electrode assembly is disposed along a single projection 140). In other instances, flexible electrode assemblies 130 may bridge across more than one projection 140 (e.g., the main body of an entire flexible electrode assembly extends along more than one projection 140).

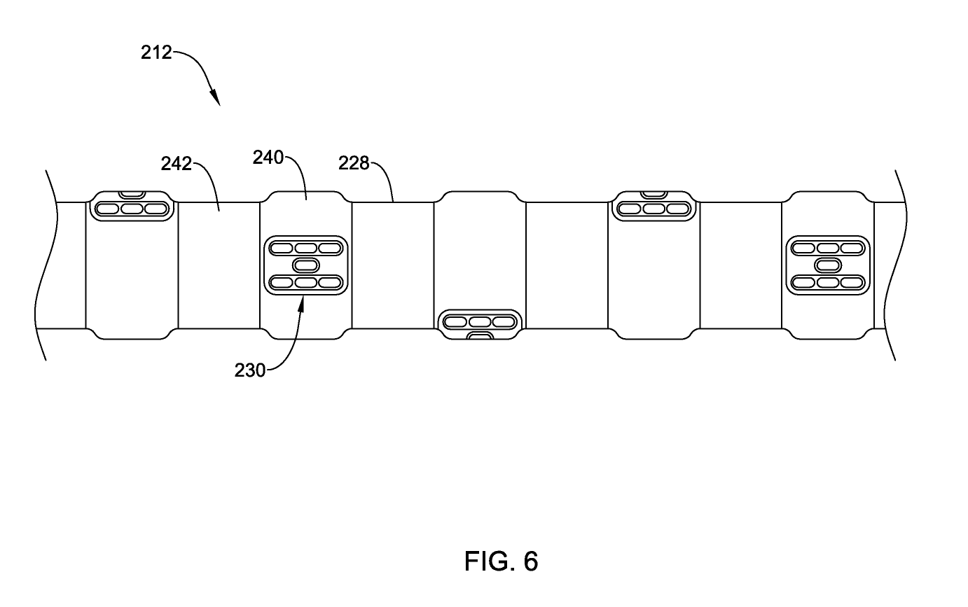

The shape, arrangement, and/or form of projections 140 may vary. For example, FIG. 6 illustrates a portion of another example device 212, similar in form and function to other devices disclosed herein, that includes balloon 228 having projections 240 formed therein. Flexible electrode assemblies 230 (shown schematically in FIG. 6, but may be similar to other electrode assemblies disclosed herein) may be coupled to projections 240. In this example, projections 240 take the form of a plurality of rings. Cooling pathways 242 may be defined between rings 240.

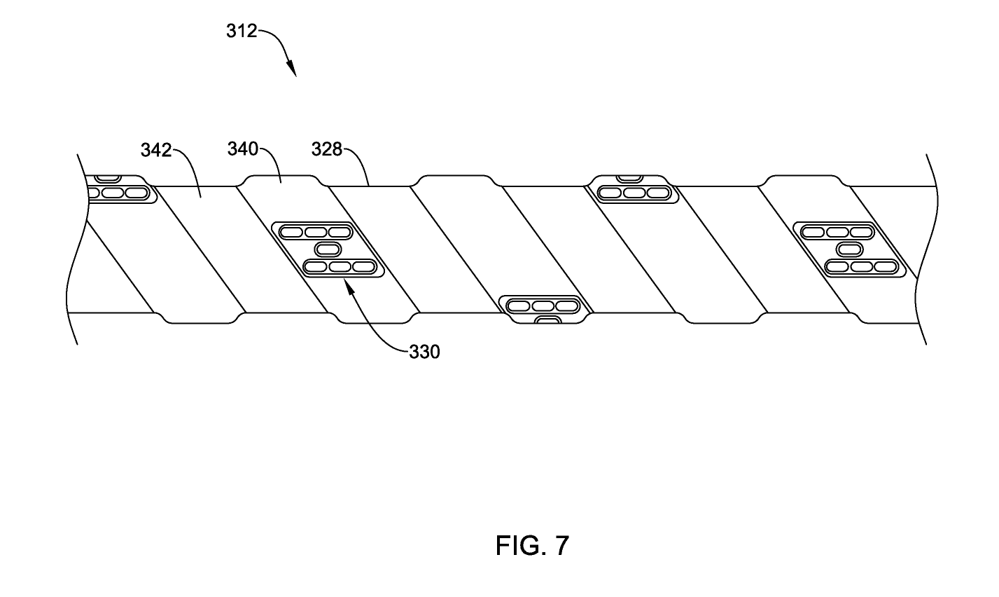

FIG. 7 illustrates a portion of another example device 312, similar in form and function to other devices disclosed herein, that includes balloon 328 having projections 340 formed therein. Flexible electrode assemblies 330 (shown schematically in FIG. 7, but may be similar to other electrode assemblies disclosed herein) may be coupled to projections 340. In this example, projections 340 take the form of a helical ridge.

Cooling pathway 342 may be defined along the helical ridge 340.

The materials that can be used for the various components of device 12 (and/or other devices disclosed herein) may include metals, metal alloys, polymers, metal-polymer composites, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also can be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or all of device 12 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of device 12 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of device 12 to achieve the same result.

In some embodiments, a degree of Magnetic Resonance Imaging (MRI) compatibility is imparted into device 12. For example, device 12, or portions thereof, may be made of a material that does not substantially distort the image and create substantial artifacts (e.g., gaps in the image). Certain ferromagnetic materials, for example, may not be suitable because they may create artifacts in an MRI image. Device 12, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A medical device, comprising:
an elongate shaft having a distal region;
an expandable member coupled to the distal region, the expandable member being capable of shifting between a first configuration and an expanded configuration;
wherein the expandable member has a distal portion, a proximal portion, and a body portion disposed between the distal portion and the proximal portion;
wherein the body portion has a projection formed therein that projects radially outward from the body portion; and
a flexible electrode assembly coupled to the projection.

2. The medical device of claim 1, wherein the expandable member includes an expandable balloon.

3. The medical device of claim 1, wherein the projection includes a circumferential ring disposed along the expandable member.

4. The medical device of claim 1, wherein the projection includes a helical ridge disposed along the expandable member.

5. The medical device of claim 1, further comprising one or more additional projections.

6. The medical device of claim 5, wherein at least some of the one or more additional projections include an additional flexible electrode assembly.

7. The medical device of claim 1, wherein the flexible electrode assembly includes a monopolar electrode.

8. The medical device of claim 1, wherein the flexible electrode assembly includes a pair of bipolar electrodes.

9. The medical device of claim 1, wherein the flexible electrode assembly includes a temperature sensor.

10. The medical device of claim 1, wherein a section of the expandable member is removed to define the projection.

11. The medical device of claim 1, wherein the projection is defined by a layer of material disposed on the expandable member.

12. A medical device, comprising:
an elongate shaft having a distal region;
an expandable balloon coupled to the distal region, the balloon being capable of shifting between a first configuration and an expanded configuration;
wherein the balloon has a distal cone portion, a proximal cone portion, and a body portion disposed between the distal cone portion and the proximal cone portion;
wherein the body portion has a projection region therein that projects radially outward from the body portion; and
a flexible electrode assembly coupled to the projection region.

13. The medical device of claim 12, wherein the projection region includes one or more circumferential rings disposed along the balloon.

14. The medical device of claim 12, wherein the projection region includes a helical ridge disposed along the balloon.

15. The medical device of claim 12, further comprising one or more additional projection regions disposed along the body portion of the balloon.

16. The medical device of claim 15, wherein at least some of the one or more additional projection regions includes an additional flexible electrode assembly.

17. The medical device of claim 12, wherein the flexible electrode assembly includes one or more monopolar electrodes.

18. The medical device of claim 12, wherein the flexible electrode assembly includes one or more pairs of bipolar electrodes.

19. The medical device of claim 12, wherein the flexible electrode assembly includes a temperature sensor.

20. A method for ablating tissue adjacent to a renal artery, the method comprising:
advancing an ablation catheter through a blood vessel to a position adjacent to a renal artery, the ablation catheter including:
an elongate shaft having a distal region,
an expandable balloon coupled to the distal region, the balloon being capable of shifting between a first configuration and an expanded configuration,
wherein the balloon has a distal cone portion, a proximal cone portion, and a body portion disposed between the distal cone portion and the proximal cone portion,
wherein the body portion has a projection region therein that projects radially outward from the body portion, and
a flexible electrode assembly coupled to the projection region;
shifting the balloon to the expanded configuration; and
activating the flexible electrode assembly.
